(19) United States
(12) Patent Application Publication
(10) Pub. No.: US 2025/0161191 A1
(43) Pub. Date: May 22, 2025

(54) DIRECT EMULSION COMPRISING A UV-SCREENING AGENT, A LIPOPHILIC ACRYLIC POLYMER, A FATTY ACID ESTER OF A POLYOL AND A CARBOXYLIC ANIONIC SURFACTANT

(71) Applicant: L'OREAL, Paris (FR)

(72) Inventors: Frédéric SIMONET, Chevilly la Rue (FR); Nideka ABEYSEKERA, Chevilly la Rue (FR); Widad AJILI, Chevilly la Rue (FR); Mahassine SAFOUANE, Chevilly la Rue (FR); Alexandra CALCAVECCHIA, Chevilly la Rue (FR)

(73) Assignee: L'OREAL, Paris (FR)

(21) Appl. No.: 18/832,780

(22) PCT Filed: Apr. 14, 2023
(86) PCT No.: PCT/EP2023/059841

§ 371 Date: Jul. 24, 2024

(30) Foreign Application Priority Data
Apr. 15, 2022 (FR) FR2203547
Mar. 16, 2023 (FR) FR2302458

(51) Int. Cl. A61K 8/81; A61K 8/06; A61K 8/9789; A61Q 17/04

(52) U.S. Cl. CPC A61K 8/8152; A61K 8/062; A61K 8/9789; A61Q 17/04

(57) ABSTRACT

The present invention relates to a composition, in particular a cosmetic or dermatological composition, in the form of an oil-in-water emulsion comprising:
at least one UV-screening agent;
at least one polymer comprising monomer units of formulae (A) and (B):

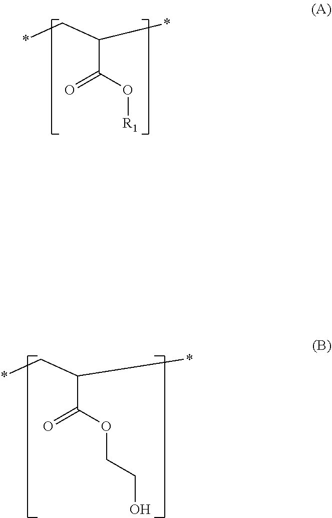

in which:
R₁, independently at each instance, is chosen from alkyl or alkenyl radicals, and
at least 60% by weight of the R₁ groups are radicals chosen from stearyl and behenyl radicals, the percentage by weight relating to the sum of all the R₁ groups present in the polymer,
and
the weight ratio of the sum of all the hydroxyethyl acrylate units to the sum of all the acrylate units bearing the R₁ group ranges from 1:30 to 1:1;
and the sum of the total of units A and B is at least 95% by weight relative to the total weight of the polymer,
the polymer having a number-average molecular weight Mn ranging from 2000 to 9000 g/mol;
at least one C₁₂-C₂₄ fatty acid ester of a C₂-C₂₄ polyol; and
at least one carboxylic or carboxylate anionic surfactant chosen from amino acids modified with at least one C₈-C₃₀ hydrocarbon-based chain, and salts thereof.

The present invention makes it possible to obtain a composition with a strong anti-UV protective power, which is stabilized over time, and which exhibits moreover good cosmetic properties, such as a refreshing capacity and a non-greasy and non-tacky texture.

The present invention relates to a composition, in particular a cosmetic or dermatological composition, in the form of an oil-in-water emulsion, comprising at least one UV-screening agent, at least one suitably selected lipophilic acrylic polymer, at least one C₁₂-C₂₄ fatty acid ester of a C₂-C₂₄ polyol, and at least one carboxylic or carboxylate anionic surfactant chosen from amino acids modified with at least one C₈-C₃₀ hydrocarbon-based chain and salts thereof, and to the use of said composition in the cosmetic and dermatological fields, in particular for caring for, treating keratin materials, and in particular for caring for, protecting and/or making up bodily or facial skin.

It is known that light radiation with wavelengths of between 280 and 400 nm makes it possible to brown the human epidermis. Rays with wavelengths more particularly between 280 and 320 nm, known as UVB rays, cause skin erythema and burns which can be detrimental to the development of a natural tan.

For these reasons, and also for aesthetic reasons, there is a constant demand for means for controlling this natural tanning in order thus to control the colour of the skin: UVB radiation should thus be screened out. It is also known that UVA rays, with wavelengths of between 320 and 400 nm, which cause browning of the skin, are capable of bringing about a detrimental change in the latter, in particular in the case of sensitive skin or of skin continually exposed to solar radiation. UVA rays cause in particular a loss in the elasticity of the skin and the appearance of wrinkles, resulting in premature skin ageing.

Thus, for aesthetic and cosmetic reasons, such as the preservation of the natural elasticity of the skin, for example, more and more people wish to control the effect of UVA rays on their skin. It is therefore desirable also to screen out UVA radiation.

For the purpose of protecting the skin and keratin materials against UV radiation, photoprotective compositions comprising organic screening agents that are active in the UVA range and in the UVB range are generally used.

Many cosmetic compositions intended for photoprotection of the skin have been proposed to date. These compositions generally contain, in an emulsified liquid support (preferably an oil-in-water emulsion), one or more organic molecules, capable of absorbing ultraviolet radiation, which are soluble in the oily and/or aqueous phase. The use of mineral pigments of a metal oxide, such as titanium dioxide, in such anti-sun compositions is increasingly frequent since these particles, which are invisible to the naked eye due to their small size, make it possible to increase the protection factor of the compositions containing same.

One of the major drawbacks of these photoprotective emulsions containing organic screening agents and/or mineral screening agents lies in the difficulty in reconciling good product stability, easy product application, effective protection, preferably at least SPF of 40 and PPD of 10, and a fresh and non-tacky feel.

The applicant has discovered that this objective can be achieved with a combination of a suitably selected emulsifying system and a suitably selected lipophilic acrylic polymer.

Thus, according to a first aspect, the present invention relates to a composition, in particular a cosmetic or dermatological composition, in the form of an oil-in-water emulsion, comprising:
at least one UV-screening agent;
at least one lipophilic acrylic polymer as defined below;
at least one C₁₂-C₂₄ fatty acid ester of a C₂-C₂₄ polyol; and
at least one carboxylic or carboxylate anionic surfactant chosen from amino acids modified with at least one C₈-C₃₀, preferably C₈-C₂₄, hydrocarbon-based chain, and salts thereof.

According to one particular embodiment, the pH of the composition in accordance with the invention is greater than or equal to 6.

Against all expectations, the inventors have noted that the use, in a photoprotective composition, of the combination of an emulsifying system comprising a fatty acid ester of a polyol and an amino acid modified with at least one fatty chain or a salt thereof with a suitably selected lipophilic acrylic polymer makes it possible to obtain a composition which is easy to spread, which has a high photoprotective power and which is nevertheless stabilized over time, having moreover good cosmetic properties such as a refreshing capacity and a non-fatty and non-tacky texture, these technical effects being further improved in a specific pH range.

Specifically, when the pH of the composition is above or equal to 6, the viscosity of the composition is significantly reduced, the composition has a greater physico-chemical stability and the sun protection obtained is greater, notably with SPF and UVAPF in vitro values that are high and robust, i.e. reproducible from one production to another.

Another subject of the invention, according to another of its aspects, is the use of a composition as defined above, for caring for keratin materials, in particular bodily and/or facial skin.

Another subject of the invention, according to yet another of its aspects, is a non-therapeutic cosmetic process for making up and/or caring for keratin materials, in particular the bodily and/or facial skin, comprising at least the application to said keratin materials of a cosmetic composition as defined above.

The invention also relates to a non-therapeutic cosmetic process for limiting the darkening of the skin and/or improving the colour and/or uniformity of the complexion, comprising the application, to the surface of the keratin material, of at least one cosmetic composition as defined previously.

The invention also relates to a non-therapeutic cosmetic process for preventing and/or treating the signs of ageing of a keratin material, comprising the application, to the surface of the keratin material, of at least one cosmetic composition as defined previously.

The composition in accordance with the invention shows good stability. This stability can be evaluated macroscopically and/or microscopically, after storage for 24 hours, one week, one month, or two months, at ambient temperature (25° C.), at 4° C., at 45° C. or at 55° C. A stable composition generally maintains its comfort and its sensory signature on application over time. More specifically, the stability of a composition can be evaluated qualitatively for example by the absence of any phase-separation phenomenon or appearance of crystals, or quantitatively through the monitoring of the change in parameters such as the viscosity or the pH.

In the context of the invention, the screening efficiency is evaluated on the basis of the evaluation of the SPF and of the UVAPF in vitro.

For the purposes of the present invention, the term “SPF” means: the sun protection factor, which measures the level of protection against UVB radiation. The value of the SPF corresponds to the ratio between the minimum time necessary to obtain sunburn with a photoprotective composition and the minimum time without product. More specifically, the term “SPF” is defined in the article A new substrate to measure sunscreen protection factors throughout the ultraviolet spectrum, J. Soc. Cosmet. Chem., 40, 127-133 (May/June 1989).

Evaluation of the SPF (Sun Protection Factor) can be carried out in vitro with a Labsphere® spectrophotometer. The plate is the material to which the photoprotective composition is applied. For this protocol, poly(methyl methacrylate) (PMMA) plates proved to be ideal. A particular protocol, presented as an example, is in the course of ISO certification under the name ISO Committee Draft 23675.

Evaluation of the Sun Protection Factor (SPF) of the compositions can also be performed in vivo according to the protocol ISO 24444:2019 “Cosmetics-Sun protection test methods-in-vivo determination of the sun protection factor (SPF)”.

For the purposes of the present invention, the term “UVAPF” is intended to mean the index characterizing the protection with respect to UVA radiation. In particular, this index can be measured in vivo according to the “PPD” (Persistent Pigment Darkening) method, ISO-24442:2022 protocol, and measures the colour of the skin observed 2 to 4 hours after exposure to UVA radiation. The evaluation of the protection with respect to UVA radiation can also be measured in vitro with a Labsphere® spectrophotometer. The plate is the material to which the solar composition is applied. For this protocol, poly(methyl methacrylate) (PMMA) plates proved to be ideal. The ISO 24443:2021 protocol describes such an in vitro method.

Other features, aspects and advantages of the invention will become apparent on reading the detailed description which follows.

The composition according to the invention is intended for topical application and thus contains a physiologically acceptable medium. The term “physiologically acceptable medium” means here a medium that is compatible with keratin materials.

In the context of the present invention, the term “keratin material” is understood to mean in particular the skin, scalp, keratin fibres, such as the eyelashes, eyebrows, head hair and body hair, nails, mucous membranes, such as the lips, and more particularly the skin and mucous membranes (body, face, area around the eyes, eyelids, lips, preferably body, face and lips).

In that which will follow and unless otherwise indicated, the limits of a range of values are included in this range, in particular in the expressions “of between” and “ranging from . . . to . . . ”.

Moreover, the expressions “at least one” and “at least” used in the present description are equivalent respectively to the expressions “one or more” and “greater than or equal to”.

According to the invention, the term “preventing” or “prevention” is intended to mean reducing the risk of occurrence or slowing down the occurrence of a given phenomenon, namely, according to the present invention, the signs of ageing of a keratin material.

The term “organic UVA-screening agent” is intended to mean any organic chemical molecule capable of absorbing at least UVA rays in the wavelength range of between 320 and 400 nm; it being possible for said molecules to likewise also absorb UVB rays in the wavelength range of between 280 and 320 nm.

The term “organic UVB-screening agent” is understood to mean any organic chemical molecule capable of absorbing exclusively UVB radiation in the wavelength range of between 280 and 320 nm.

The term “emulsion” is intended to mean any macroscopically homogeneous, kinetically stable composition comprising at least two mutually immiscible phases; one being the dispersing continuous phase and the other being dispersed in said continuous phase in the form of droplets. The two phases are kinetically stabilized by at least one emulsifying system generally comprising at least one emulsifying surfactant.

A distinction is made between emulsions of oil-in-water type, termed “direct”, constituted of a continuous aqueous dispersing phase and of a non-continuous oily dispersed phase, and emulsions of water-in-oil type, termed “inverse”, constituted of a continuous oily dispersing phase and of a non-continuous aqueous dispersed phase. Multiple emulsions, such as water-in-oil-in-water or oil-in-water-in-oil, also exist.

The compositions in accordance with the invention are direct emulsions.

### UV-Screening Agents

The compositions according to the invention contain one or more UV-screening agents chosen from hydrophilic, lipophilic or insoluble organic UV-screening agents and/or one or more mineral pigments. Preferentially, the screening system will be constituted of at least one hydrophilic, lipophilic or insoluble organic UV-screening agent.

The term “hydrophilic UV-screening agent” is intended to mean any cosmetic or dermatological organic or inorganic compound for screening out UV radiation, which can be fully dissolved in molecular form in a liquid aqueous phase or else which can be dissolved in colloidal form (for example in micellar form) in a liquid aqueous phase.

The term “lipophilic screening agent” is intended to mean any cosmetic or dermatological organic or inorganic compound for screening out UV radiation, which can be fully dissolved in molecular form in a liquid fatty phase or else which can be dissolved in colloidal form (for example in micellar form) in a liquid fatty phase.

The term “insoluble UV-screening agent” is intended to mean any cosmetic or dermatological organic or inorganic compound for screening out UV radiation which has a solubility in water of less than 0.5% by weight and a solubility of less than 0.5% by weight in the majority of organic solvents such as liquid paraffin, fatty alcohol benzoates and fatty acid triglycerides, for example Miglyol 812® sold by Dynamit Nobel. This solubility, determined at 70° C., is defined as the amount of product in solution in the solvent at equilibrium with an excess of solid in suspension after returning to ambient temperature. It can be easily evaluated in the laboratory.

The organic UV-screening agents are chosen in particular from cinnamic compounds; anthranilate compounds; salicylic compounds; dibenzoylmethane compounds; benzylidenecamphor compounds; benzophenone compounds; β,β-diphenylacrylate compounds; triazine compounds; benzotriazole compounds; benzalmalonate compounds, in particular those cited in patent U.S. Pat. No. 5,624,663; benzimidazole derivatives; imidazoline compounds; bis-benzazolyl compounds, such as described in patents EP 669 323 and U.S. Pat. No. 2,463,264; methylenebis(hydroxyphenylbenzotriazole) compounds, such as described in applications U.S. Pat. Nos. 5,237,071, 5,166,355, GB 2 303 549, DE 197 26 184 and EP 893 119; benzoxazole compounds, such as described in patent applications EP 0 832 642, EP 1 027 883, EP 1 300 137 and DE 101 62 844; screening polymers and screening silicones, such as those described in particular in application WO 93/04665; dimers derived from α-alkylstyrene, such as those described in patent application DE 198 55 649; 4,4-diarylbutadiene compounds, such as described in applications EP 0 967 200, DE 197 46 654, DE 197 55 649, EP-A-1 008 586, EP 1 133 980 and EP 133 981, and mixtures thereof.

Mention may be made, as examples of organic photoprotective agents, of those denoted below under their INCI name and/or their chemical name.

### Cinnamic Compounds:

Ethylhexyl methoxycinnamate, sold especially under the trade name Parsol® MCX by DSM Nutritional Products,
Isoamyl p-Methoxycinnamate sold under the trade name Neo Heliopan E 1000® by Symrise,

### Dibenzoylmethane Compounds:

Butyl Methoxydibenzoylmethane, sold in particular under the trade name Parsol® 1789 by DSM Nutritional Products,

### Salicylic Compounds:

Homosalate sold under the name Parsol® HMS by DSM Nutritional Products,
Ethylhexyl salicylate sold under the name Neo Heliopan® OS by Symrise,

### β,β-Diphenylacrylate Compounds:

Octocrylene, sold in particular under the trade name Uvinul® N 539 T by BASF,

### Benzophenone Compounds:

Benzophenone-3 or Oxybenzone, sold under the trade name Uvinul® M 40 by BASF,
n-Hexyl 2-(4-diethylamino-2-hydroxybenzoyl)benzoate, having the INCI name Diethylamino hydroxybenzoyl hexyl benzoate sold under the trade name Uvinul® A Plus or, as a mixture with ethylhexyl methoxycinnamate, under the trade name Uvinul® A Plus B by BASF,
1,1′-(1,4-Piperazinediyl)bis [1-[2-[4-(diethylamino)-2-hydroxybenzoyl]phenyl]metha none] (CAS 919803-06-8), as described in application WO 2007/071584; this compound advantageously being used in micronized form (mean size of 0.02 to 2 μm), which may be obtained, for example, according to the micronization process described in applications GB-A-2 303 549 and EP-A-893 119, and in particular in the form of an aqueous dispersion,

### Benzylidenecamphor Compounds:

4-Methylbenzylidenecamphor sold under the name Eusolex® 6300 by Merck,
Terephthalylidenedicamphorsulfonic acid manufactured under the name Mexoryl® SX by Noveal,

### Phenylbenzimidazole Compounds:

Phenylbenzimidazolesulfonic acid sold especially under the trade name Eusolex® 232 by Merck,

### Bis-Benzazolyl Compounds:

Disodium phenyl dibenzimidazole tetrasulfonate sold under the trade name Neo Heliopan® AP by Symrise,

### Phenylbenzotriazole Compounds:

Drometrizole trisiloxane, manufactured under the name Mexoryl® XL by Noveal,

### Methylenebis(Hydroxyphenylbenzotriazole) Compounds:

Methylenebis(benzotriazolyl)tetramethylbutylphenol, in particular in solid form, such as the product sold under the trade name Mixxim BB/100® by Fairmount Chemical, or in the form of an aqueous dispersion of micronized particles with a mean particle size ranging from 0.01 to 5 μm, more preferentially from 0.01 to 2 μm and more particularly from 0.020 to 2 μm, with at least one alkyl polyglycoside surfactant having the structure CₙH₂ₙ₊₁O(C₆H₁₀O₅)ₓH, in which n is an integer from 8 to 16 and x is the mean degree of polymerization of the (C₆H₁₀O₅) unit and ranges from 1.4 to 1.6, as described in patent GB-A-2 303 549, sold in particular under the trade name Tinosorb® M by BASF, or in the form of an aqueous dispersion of micronized particles with a mean particle size ranging from 0.02 to 2 μm, more preferentially from 0.01 to 1.5 μm and more particularly from 0.02 to 1 μm, in the presence of at least one polyglyceryl mono(C₈-C₂₀)alkyl ester with a degree of glycerol polymerization of at least 5, such as the aqueous dispersions described in application WO 2009/063392, in particular the product sold under the name Tinosorb® WPGL by BASF,

### Triazine Compounds:

3,3′-(1,4-Phenylene)bis(5,6-diphenyl-1,2,4-triazine), with the INCI name Phenylene Bis-Diphenyltriazine,
Bis-Ethylhexyloxyphenol Methoxyphenyl Triazine sold under the trade name Tinosorb® by BASF, and in its water-dispersible form having the INCI name Bis-Ethylhexyloxyphenol Methoxyphenyl Triazine (and) Acrylates/C₁₂-22 Alkyl Methacrylate Copolymer, under the trade name Tinosorb® S LiteAqua by BASF,
Ethylhexyl Triazone, sold in particular under the trade name Uvinul® T 150 by BASF,
Diethylhexyl Butamido Triazone, sold under the trade name Uvasorb® HEB by 3V Sigma,
symmetrical triazine screening agents substituted by naphthalenyl groups or polyphenyl groups described in patent U.S. Pat. No. 6,225,467, application WO2004/085412 (see compounds 6 and 9) or the document “Symmetrical Triazine Derivatives”, IP.COM IPCOM000031257 Journal, INC West Henrietta, NY, US (20 Sep. 2004), in particular 2,4,6-tris(biphenyl)triazine and 2,4,6-tris(terphenyl)triazine sold under the name Tinosorb® A2B by BASF and which is mentioned in patent applications WO06/035000, WO06/034982, WO06/034991, WO06/035007, WO2006/034992 and WO2006/034985, these compounds advantageously being used in micronized form (mean particle size of 0.02 to 3 μm), which can be obtained, for example, according to the micronization process described in applications GB-A-2 303 549 and EP-A-893 119, and in particular in aqueous dispersion form,

### Anthranilic Compounds:

Menthyl anthranilate sold under the trade name Neo Heliopan® MA by Symrise,

### Benzalmalonate Compounds:

Polyorganosiloxane bearing benzalmalonate functions, for instance Polysilicone-15, sold under the trade name Parsol® SLX by DSM Nutritional Products,

### Benzoxazole Compounds:

2,4-Bis [5-1 (dimethylpropyl)benzoxazol-2-yl-(4-phenyl)imino]-6-(2-ethylhexyl) imin o-1,3,5-triazine, sold under the name Uvasorb®K2A by 3V Sigma,
2-[4-(1,3-benzoxazol-2-yl)phenyl]-1,3-benzoxazole, having the CAS number 904-39-2.

The preferential organic screening agents are chosen from:
Ethylhexyl Methoxycinnamate,
Ethylhexyl Salicylate,
Homosalate,
Butyl Methoxydibenzoylmethane,
Octocrylene,
Phenylbenzimidazole Sulfonic Acid,
Benzophenone-3,
Diethylamino hydroxybenzoyl hexyl benzoate,
4-Methylbenzylidene camphor,
Terephthalylidene Dicamphor Sulfonic Acid,
Disodium Phenyl Dibenzimidazole Tetrasulfonate,
Methylene Bis-Benzotriazolyl Tetramethylbutylphenol,
Bis-Ethylhexyloxyphenol Methoxyphenyl Triazine,
Ethylhexyl Triazone,
Diethylhexyl Butamido Triazone,
2,4,6-Tris(biphenyl)triazine,
2,4,6-Tris(terphenyl)triazine,
Drometrizole Trisiloxane,
Polysilicone-15,
Bis(diethylaminohydroxybenzoyl benzoyl) piperazine,
and mixtures thereof.
The particularly preferred organic screening agents are chosen from:
Ethylhexyl Salicylate,
Homosalate,
Butyl Methoxydibenzoylmethane,
Octocrylene,
Diethylamino hydroxybenzoyl hexyl benzoate,
Terephthalylidene Dicamphor Sulfonic Acid,
Bis-Ethylhexyloxyphenol Methoxyphenyl Triazine,
Ethylhexyl Triazone,
Diethylhexyl Butamido Triazone,
Bis(diethylaminohydroxybenzoyl benzoyl) piperazine,
Drometrizole Trisiloxane,
2,4,6-Tris(biphenyl)triazine,
and mixtures thereof.

The inorganic UV-screening agents used in accordance with the present invention are metal oxide pigments. More preferentially, the inorganic UV-screening agents of the invention are metal oxide particles having a mean elementary particle size of less than or equal to 0.5 μm, more preferentially of between 0.005 and 0.5 μm, even more preferentially of between 0.01 and 0.2 μm, even better still between 0.01 and 0.1 μm and more particularly between 0.015 and 0.05 μm. They are in particular described in annex VI, updated on 22 Sep. 2021, of EU Regulation No 1223/2009 on cosmetic products, but are not limited to this list.

They can be chosen in particular from titanium oxide, zinc oxide, iron oxide, zirconium oxide, cerium oxide or mixtures thereof.

Such coated or uncoated metal oxide pigments are described in particular in patent application EP-A-0 518 773. As commercial pigments, mention may be made of the products sold by Croda, Tayca and Merck.

The metal oxide pigments may be coated or uncoated.

The coated pigments are pigments which have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminium salts of fatty acids, metal alkoxides (of titanium or aluminium), polyethylene, silicones, proteins (collagen, elastin), alkanolamines, silicon oxides, metal oxides or sodium hexametaphosphate.

The coated pigments are more particularly titanium oxides coated:
with silica hydrate, such as the product MT-100WP from Tayca,
with silica and iron oxide, such as the product Sunveil F® from Ikeda,
with silica and alumina, such as the products MT-500SA® and MT-100SA® from Tayca and Tioveil™ AQ-N from Croda,
with alumina, such as the product TTO-55 (A)® from Ishihara,
with alumina and aluminium stearate, such as the products MT-100TV®, MT-100Z® and MT-01® from Tayca, the product Solaveil™ CT100 from Croda and the product Eusolex T-AVO® from Merck,
with silica, alumina and alginic acid, such as the product MT-100AQ® from Tayca,
with alumina and aluminium laurate,
with iron oxide and iron stearate,
with zinc oxide and zinc stearate,
with silica and alumina and treated with a silicone, such as the products MTY-500SAS® or Microtitanium Dioxide MT-100SAS® from Tayca,
with silica, alumina, and aluminium stearate and treated with a silicone,
with silica and treated with a silicone,
with alumina and treated with a silicone, such as the product TTO-55 (S)® from Ishihara,
with triethanolamine,
with stearic acid, such as the product TTO-55 (C)® from Ishihara,
with sodium hexametaphosphate,
TiO₂ treated with octyltrimethylsilane,
TiO₂ treated with a polydimethylsiloxane,
anatase/rutile TiO₂ treated with a polydimethylhydrogenosiloxane,
TiO₂ coated with triethylhexanoin, with aluminium stearate and with alumina sold under the trade name Solaveil™ CT-200 by Croda,
TiO₂ coated with aluminium stearate, with alumina and with silicone, sold under the trade name Solaveil™ CT-12W by Croda,
TiO₂ coated with lauroyl lysine,
TiO₂ coated with C₉-C₁₅ fluoroalcohol phosphate and with aluminium hydroxide.

Mention may also be made of TiO₂ pigments doped with at least one transition metal such as iron, zinc or manganese and more particularly manganese. Preferably, said doped pigments are in the form of an oily dispersion. The oil present in the oily dispersion is preferably chosen from triglycerides, including those of capric/caprylic acids. The oily dispersion of titanium oxide particles can additionally comprise one or more dispersing agents, such as, for example, a sorbitan ester, such as sorbitan isostearate, a polyoxyalkylenated glycerol fatty acid ester, such as Tri-PPG-3 Myristyl Ether Citrate and Polyglyceryl-3 Polyricinoleate. Preferably, the oily dispersion of titanium oxide particles comprises at least one dispersing agent chosen from polyoxyalkylenated glycerol fatty acid esters. Mention may be made more particularly of the oily dispersion of TiO₂ particles doped with manganese in capric/caprylic acid triglyceride in the presence of Tri-PPG-3 myristyl ether citrate and polyglyceryl-3 polyricinoleate and sorbitan isostearate having the INCI name: titanium dioxide (and) Tri-PPG-3 myristyl ether citrate (and) polyglyceryl-3 ricinoleate (and) sorbitan isostearate, for instance the product sold under the trade name Optisol™ OTP-1 by Croda.

The uncoated titanium oxide pigments are for example sold by Tayca under the trade names MT-500B or MT-600B®, or by Evonik under the name Degussa P 25.

The uncoated zinc oxide pigments are, for example:
those sold under the name Z-Cote® by BASF;
those sold under the name NanoArc® Zinc Oxide by Nanophase Technologies.

The coated zinc oxide pigments are, for example:
—ZnO coated with polymethylhydrosiloxane;
Solaveil™ CZ-100 from Croda, dispersed in C₁₂-C₁₅ alkyl benzoate (INCI: Zinc Oxide (and) C₁₂-15 Alkyl Benzoate (and) Polyhydroxystearic Acid (and) Isostearic Acid);
those sold under the name Daitopersion Zn-60 VAR by Daito Kasei (dispersions in C₉-C₁₂ alkane with a dispersant);
those sold under the name SPD-Z5® by Shin-Etsu (ZnO coated with silicone-grafted acrylic polymer, dispersed in cyclodimethylsiloxane).

The uncoated cerium oxide pigments may be, for example, those sold under the name Rhodigard® W185 by Solvay.

Mention may also be made of mixtures of metal oxides, especially of titanium dioxide and cerium dioxide, including the mixture in equal weights of titanium dioxide and cerium dioxide, coated with silica, and also the mixture of titanium dioxide and zinc dioxide coated with alumina, silica and silicone or coated with alumina, silica and glycerol.

According to the invention, coated or uncoated titanium oxide pigments are particularly preferred.

The UV-screening agents can be present in the composition according to the invention in a content ranging from 0.1% to 60% by weight, preferably from 1% to 40% by weight, and even more preferentially from 5% to 30% by weight, and better still from 10% to 25% by weight, relative to the total weight of the composition.

### Lipophilic Acrylic Polymer

The lipophilic acrylic polymer present in the composition in accordance with the invention comprises monomer units of formulae (A) and (B):

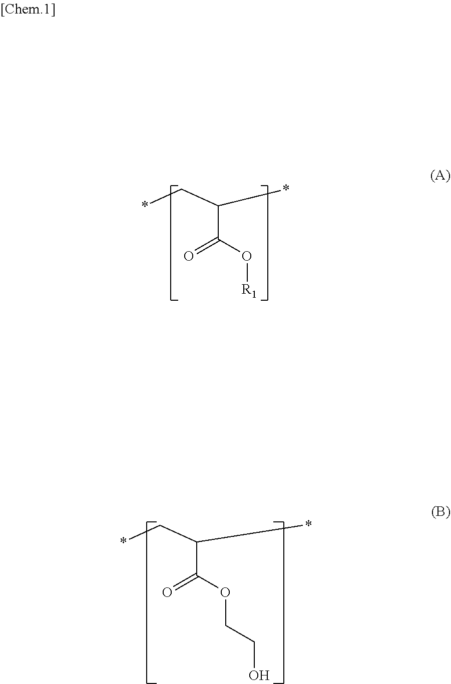

in which:
R₁, independently at each instance, is chosen from alkyl or alkenyl radicals, and
at least 60% by weight of the R₁ groups are radicals chosen from stearyl and behenyl radicals, the percentage by weight relating to the sum of all the R₁ groups present in the polymer, and
the weight ratio of the sum of all the hydroxyethyl acrylate units to the sum of all the acrylate units bearing the R₁ group ranges from 1:30 to 1:1,
and the sum of the total of units A and B is at least 95% by weight relative to the total weight of the polymer.

Preferably, R₁ is constituted of alkyl radicals, preferably of C₁₆-C₂₂ alkyl radicals, and more preferentially of stearyl (C₁₈) radicals or of behenyl (C₂₂) radicals.

Preferably, at least 70% by weight of the R₁ groups are stearyl or behenyl radicals, preferentially at least 80% by weight and more preferentially at least 90% by weight.

According to one preferred embodiment, all the R₁ groups are behenyl radicals.

According to another preferred embodiment, all the R₁ groups are stearyl radicals.

Preferably, the weight ratio of the sum of all the hydroxyethyl acrylate units to the sum of all the acrylate units bearing the R₁ group ranges from 1:15 to 1:1, preferably ranges from 1:10 to 1:4.

Advantageously, the polymer units present in the polymer are constituted of the units (A) and (B) previously described.

The polymer has a number-average molecular weight Mn ranging from 2000 to 9000 g/mol, preferably ranging from 5000 to 9000 g/mol. The number-average molecular weight may be measured via the gel permeation chromatography method, for example according to the method described in the example hereinbelow.

Preferably, the polymer has a melting point ranging from 40° C. to 70° C. and preferentially ranging from 45° C. to 67° C. The melting point is measured by differential scanning calorimetry (DSC), for example according to the method described in the example hereinbelow.

According to a first embodiment, when the polymer is such that at least 60% by weight of the R₁ groups are stearyl radicals, then the polymer preferably has a melting point ranging from 40° C. to 60° C., and preferentially ranging from 45° C. to 55° C.

According to a second embodiment, when the polymer is such that at least 60% by weight of the R₁ groups are behenyl radicals, then the polymer has a melting point ranging from 60° C. to 70° C., and preferentially ranging from 63° C. to 67° C.

The polymer used according to the invention can be prepared by polymerization of a monomer having the formula below:

CH₂═CH—COO—R₁,
R₁ having the meaning previously described, and of 2-hydroxyethyl acrylate.

The polymerization may be performed according to known methods, such as solution polymerization or emulsion polymerization.

The polymerization is, for example, described in document US 2007/0264204.

The lipophilic acrylic polymer can be present in the composition according to the invention in a content of active material ranging from 0.05 to 10% by weight, relative to the total weight of the composition, preferably ranging from 0.1 to 5% by weight and better still ranging from 0.2 to 2% by weight.

C₁₂-C₂₄ Fatty Acid Esters of a C₂-C₂₄ Polyol

The composition in accordance with the invention comprises at least one C₁₂-C₂₄ fatty acid ester of a C₂-C₂₄ polyol, preferably at least one C₁₆-C₁₈ fatty acid ester of glycerol.

The C₁₂-C₂₄ fatty acid for the fatty acid ester of a polyol, preferably the fatty acid ester of glycerol, can be a saturated or unsaturated acid, for example stearic acid, oleic acid or palmitic acid.

For the purposes of the present invention, the term “polyol” should be understood as meaning any organic molecule including at least two free hydroxyl groups.

The polyol for the fatty acid ester of a polyol can be a compound of linear, branched or cyclic, saturated or unsaturated alkyl type, bearing on the alkyl chain at least two OH functions and in particular at least three OH functions.

The polyols that are advantageously suitable for the fatty acid ester of a polyol are those preferably having 3 to 16 carbon atoms.

Advantageously, the polyol may be chosen, for example, from ethylene glycol, pentaerythritol, trimethylolpropane, propylene glycol, 1,3-propanediol, butylene glycol, isoprene glycol, pentylene glycol, hexylene glycol, polyglycerin-3 and glycerol, and mixtures thereof.

According to one preferred embodiment of the invention, said polyol is chosen from 1,3-propanediol, polyglycerin-3, glycerol and pentylene glycol, and mixtures thereof. It is preferably glycerol.

According to one particular embodiment of the invention, the composition comprises at least one C₁₂-C₂₄ fatty acid ester of glycerol.

By way of examples of C₁₂-C₂₄ fatty acid esters of glycerol, mention may be made of glyceryl laurate, glyceryl oleate, glyceryl stearate, glyceryl palmitate, and a mixture thereof.

As commercially available products, mention may be made of glyceryl stearate, such as the product sold under the name Tegin MO by Evonik Goldschmidt, and glyceryl laurate, such as the product sold under the name Imwitor 3120 by Hiils.

Use may also be made of monoglyceride stearates (INCI name: Glyceryl Stearate), for example sold under the name Dimodan HP by Danisco, or those sold under the name Tegin 90 by Evonik Goldschmidt.

Preferably, the C₁₂-C₂₄ fatty acid ester of glycerol is glyceryl stearate.

Advantageously, the C₁₂-C₂₄ fatty acid ester(s) of a C₂-C₂₄ polyol is/are present in the composition in accordance with the invention in an amount ranging from 0.1% to 2% by weight, preferably ranging from 0.2% to 0.8% by weight, relative to the total weight of the composition.

Carboxylic or Carboxylate Anionic Surfactants Chosen from Fatty-Chain Amino Acids and Salts Thereof

The composition in accordance with the invention comprises at least one carboxylic or carboxylate anionic surfactant chosen from amino acids modified with at least one C₈-C₃₀, preferably C₈-C₂₄, hydrocarbon-based chain, and salts thereof.

The term “hydrocarbon-based chain” is intended to mean a linear or branched, saturated or unsaturated organic group constituted mainly of hydrogen atoms and carbon atoms, in which one or more carbon atoms can be replaced with an oxygen atom or a nitrogen atom. As examples of C₈-C₃₀ hydrocarbon-based chains, mention may be made of C₈-C₃₀ acyl radicals.

By way of examples of amino acids, mention may be made of alanine, arginine, aspartic acid, glutamic acid, glycine, isoleucine, leucine, lysine, phenylalanine, serine, tyrosine, valine and sarcosine.

More specifically, mention may be made, by way of carboxylate anionic surfactants chosen from the salts of amino acids modified with at least one fatty chain, of the following compounds: dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, cocoyl methyl β-alaninate, lauroyl β-alaninate, lauroyl methyl β-alaninate, myristoyl β-alaninate, potassium lauroyl methyl β-alaninate, sodium cocoyl alaninate, sodium cocoyl methyl β-alaninate and sodium myristoyl methyl β-alaninate palmitoyl glycinate, sodium lauroyl glycinate, sodium cocoyl glycinate, sodium myristoyl glycinate, potassium lauroyl glycinate, potassium cocoyl glycinate, potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate ammonium lauroyl sarcosinate, sodium lauroyl aspartate, sodium myristoyl aspartate, sodium cocoyl aspartate, sodium caproyl aspartate, disodium lauroyl aspartate, disodium myristoyl aspartate, disodium cocoyl aspartate, disodium caproyl aspartate, potassium lauroyl aspartate, potassium myristoyl aspartate, potassium cocoyl aspartate, potassium caproyl aspartate, dipotassium lauroyl aspartate, dipotassium myristoyl aspartate, dipotassium cocoyl aspartate, dipotassium caproyl aspartate, and mixtures thereof.

According to one particular embodiment of the invention, the carboxylic or carboxylate anionic surfactant(s) is/are chosen from the compounds having the following structure:

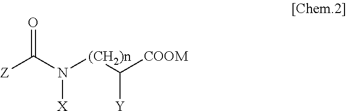

in which:
Z represents a saturated or unsaturated, linear or branched hydrocarbon-based group comprising from 8 to 24 carbon atoms, preferably from 8 to 22 carbon atoms,
X is a hydrogen atom or a methyl group;
n is equal to 0 or to 1;
Y is a group chosen from a hydrogen atom and a —CH₃, —CH(CH₃)₂, —CH₂CH(CH₃)₂, —CH(CH₃) CH₂CH₃, —CH₂C₆H₅, —CH₂C₂H₄OH, —CH₂OH, —CH(OH)CH₃, —(CH₂)₄NH₂, —(CH₂)₃NHC(NH)NH₂, —CH₂C(O)OM⁺, —(CH₂)₂C(O)OH and —(CH₂)₂C(O)O⁻M⁺ group; and
M is a hydrogen atom or a cation such as sodium, potassium, ammonium or triethanolamine.

According to one preferred embodiment, in formula 2:
Z represents a linear or branched C₈-C₂₂ alkyl or alkenyl group;
X is a hydrogen atom or a methyl radical;
n is equal to 0;
Y is a hydrogen atom, a —(CH₂)₂C(O)OH group or a —(CH₂)₂C(O)O⁻M⁺ group; and
M is a cation such as sodium, potassium, ammonium or triethanolamine.

According to one particular embodiment, the composition according to the invention comprises at least one acyl glutamic acid (INCI name: acyl glutamic acid) or a salt thereof (acyl glutamates).

Preferably, the acyl glutamic acid(s) is/are chosen from acyl glutamic acids in which the acyl group comprises from 8 to 30 carbon atoms, preferably from 10 to 24 carbon atoms and even more preferentially from 12 to 22 carbon atoms, such as, for example, lauroylglutamic acid, myristoylglutamic acid, palmitoylglutamic acid, stearoylglutamic acid, behenoylglutamic acid, olivoylglutamic acid, cocoylglutamic acid, and the salts of these acids, especially the salts of alkali metals such as Na, Li or K, preferably Na or K, the salts of alkaline-earth metals such as Mg, or the ammonium salts of said acids.

Preferably, mention may be made, by way of salts of amino acids modified with at least one C₈-C₃₀ hydrocarbon-based chain, of glutamate salts, and in particular dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate.

Preferably, the acyl glutamic acid(s) or a salt thereof is/are chosen from lauroylglutamic acids, cocoylglutamic acids, sodium stearoyl glutamate, potassium lauroyl glutamate, potassium cocoyl glutamate, sodium olivoyl glutamate and mixtures thereof.

More preferentially, the acyl glutamic acid or a salt thereof is sodium stearoyl glutamate (INCI name).

Such compounds are sold under the name Amisoft by Ajinomoto and in particular under the references Amisoft CA, Amisoft LA, Amisoft HS 11 PF, Amisoft MK-11, Amisoft LK-11 and Amisoft CK-11, or alternatively under the name Eumulgin SG by Cognis.

Mention may also be made of triethanolamine cocoyl glutamate sold under the name Amisoft CT12 by Ajinomoto, and triethanolamine lauroyl glutamate sold under the name Acylglutamate LT-12 by Ajinomoto.

As acyl glutamic acid salts, mention may also be made of sodium hydrogenated tallowoyl glutamate, such as the product sold under the reference Acylglutamate HS 11 by Ajinomoto and disodium hydrogenated tallow glutamate, such as the product sold under the reference Acylglutamate HS-21 by Ajinomoto.

Mention may also be made of commercial mixtures of surfactants comprising at least one glutamic acid derivative or a salt of said derivative, for instance the mixture of acyl glutamate salts such as Amisoft LS-22 sold by Ajinomoto.

According to one preferred embodiment of the invention, the monosodium salt of n-stearoyl-L-glutamic acid (INCI name: sodium stearoyl glutamate), such as the product sold by Ajinomoto under the reference Amisoft HS 11 PF, is used.

The amino acid(s) modified with at least one C₈-C₃₀ hydrocarbon-based chain, and salts thereof, may be present in the composition in a content ranging from 0.01% to 5% by weight, preferably from 0.01% to 2% by weight, more preferentially from 0.05% to 1% by weight, better still from 0.1% to 0.8% by weight, and even better still from 0.1% to 0.5% by weight, relative to the total weight of the composition.

### C12-C24 Saturated Fatty Alcohols

The composition in accordance with the invention can also comprise at least one C₁₂-C₂₄ saturated fatty alcohol.

According to one particular embodiment of the invention, the C₁₂-C₂₄ saturated fatty alcohols are linear. Preferably, the fatty alcohols comprise from 14 to 22 carbon atoms.

The fatty alcohol(s) that can be used in the context of the present invention can in particular be chosen from cetyl alcohol, stearyl alcohol, cetearyl alcohol, myristyl alcohol, lauryl alcohol, tridecyl alcohol, pentadecyl alcohol, hexadecyl alcohol, arachidyl alcohol and behenyl alcohol. They are preferably chosen from cetyl alcohol, stearyl alcohol and cetearyl alcohol

As cetyl alcohol that is most particularly suitable for the invention, use may for example be made of those sold under the names Ecorol® 16/98 F and Ecorol® 16/98 P by Ecogreen Oleochemicals, Tegoalkanol® 16 by Evonik Goldschmidt, Lanette® 16 by Cognis, Vegarol® 1698 by VVF, Alkonat® 1698 P by Oxiteno, Cetyl Alcohol 98% MIN by Emery Oleochemicals, Ginol® 16 (98%) by Godrej Industries, Nacol® 16-98 by Sasol, Kalcol® 6098 by Kao and Acilol® 16 by Aegis Chemical.

As stearyl alcohol that is most particularly suitable for the invention, use may for example be made of those sold under the names Tegoalkanol® 18 by Evonik Goldschmidt, Ecorol® 18/98 F and Ecorol® 18/98 P by Ecogreen Oleochemicals, Lanette® 18 by Cognis, Kalcol® 8098 by Kao, Acilol® 18 by Aegis Chemical, Nacol® 18-98 by Sasol and NAAR 45 by Nihon Yushi.

As cetylstearyl alcohols that are most particularly suitable for the invention, use may for example be made of those sold under the names Ecorol® 68/50 F and Ecorol® 68/50 P by Ecogreen Oleochemicals, Lanette® O OR and Lanette® O OR Flakes by Cognis, Alkonat® 1618 C50 P by Oxiteno, Nafol® 16-18 EN by Sasol, Alcohol Cetoestearilico 50/50 by Industria Quimica Del Centro, Conol® 30 CK by New Japan Chemical, Cetylstearyl Alcohol 50:50 by Evonik Goldschmidt, Kalcol® 6850 by Kao, Vegarol® 1618 (50:50) by VVF and Ginol® 1618 50:50 OR by Godrej Industries.

As behenyl alcohol that is most particularly suitable for the invention, use may be made, for example, of those sold under the name Lanette 22 by BASF.

The composition according to the invention has an alpha-gel structure, that is to say that it has a lamella phase, due to the presence of a surfactant and of a C₁₂-C₂₄ saturated fatty alcohol.

Advantageously, the C₁₂-C₂₄ saturated fatty alcohol(s) is/are present in the composition in an amount ranging from 0.1% to 5% by weight, preferably ranging from 0.2% to 3% by weight, and even more preferentially ranging from 0.5% to 2% by weight, relative to the total weight of the composition.

### Waxes

The composition in accordance with the invention may also comprise at least one wax.

For the purposes of the present invention, the term “wax” means a deformable or undeformable lipophilic compound, which is solid at ambient temperature (25° C.), with a reversible solid/liquid change of state, having a melting point of greater than or equal to 30° C., which may be up to 120° C. In particular, the waxes that are suitable for use in the invention may have a melting point of greater than or equal to 60° C. and in particular greater than or equal to 70° C.

The term “lipophilic compound” refers to a compound having an acid number and a hydroxyl number of less than 150 mg KOH/g.

For the purposes of the invention, the melting point corresponds to the temperature of the most endothermic peak observed on thermal analysis (DSC) as described in standard ISO 11357-3; 1999. The melting point of the wax may be measured using a differential scanning calorimeter (DSC), for example the calorimeter sold under the name MDSC 2920 by TA Instruments or the calorimeter sold under the name DSC Q100 by TA Instruments with the TA Universal Analysis software.

The measuring protocol is as follows:

A sample of 5 mg of wax placed in a crucible is subjected to a first temperature ramp passing from −20° C. to 100° C., at a heating rate of 10° C./minute, it is then cooled from 100° C. to −20° C. at a cooling rate of 10° C./minute and is finally subjected to a second temperature ramp passing from −20° C. to 100° C. at a heating rate of 5° C./minute. During the second temperature ramp, the variation in the difference in power absorbed by the empty crucible and by the crucible containing the sample of wax is measured as a function of the temperature. The melting point of the compound is the temperature value corresponding to the top of the peak of the curve representing the variation in the difference in power absorbed as a function of the temperature.

The waxes can be hydrocarbon waxes, silicone waxes and/or fluoro waxes, and can be of plant, animal, mineral and/or synthetic origin.

The wax(es) may be present in each composition in a content ranging from 0.01% to 5% by weight, preferably from 0.1% to 3% by weight and better still from 0.2% to 2% by weight, relative to the total weight of the composition.

According to one particular embodiment, the composition in accordance with the invention comprises at least one wax of plant origin.

According to one preferred embodiment, the composition in accordance with the invention comprises at least one jojoba ester wax.

The jojoba ester wax may be in an unsaturated form of jojoba esters, which may be the entirely hydrogenated jojoba ester having the formula below:

R¹—COO—CH₂—R¹ [Chem.3]
in which:
R¹ is a CH₃—(CH₂)_{y} group, y is equal to 16, 18, 20 or 22.

The jojoba ester wax can be obtained by hydrogenation of a jojoba wax ester (jojoba oil) having the formula below:

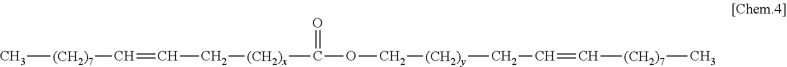

in which x and y are equal to 6, 8, 10 or 12.

The jojoba wax esters are composed of straight-chain monounsaturated fatty alcohols and of monounsaturated fatty acids. The only double bond is located in the middle (position n-9), starting from the methyl end group (—CH₃) of the chain of the respective fatty acid or alcohol.

Such wax esters are composed of fatty alcohols and of fatty esters with an even number of carbon atoms, mainly 20 and 22 carbon atoms. The resulting material comprises esters having chain lengths of 38, 40, 42 and 44 carbon atoms, with a small amount of esters of 36 and 46 carbon atoms being present. The typical composition of the wax esters is indicated below.

TABLE 1
Wax esters

| | | typical % (surface by |
|---|---|---|
| (X, Y) | Chain length | GPC) |
| (6, 6) | 36 | 1 |
| (6, 8) (8, 6) | 38 | 8 |
| (6, 10) (8, 8) (10, 6) | 40 | 39 |
| (10, 8) (8, 10) | 42 | 38 |
| (10, 10) | 44 | 13 |
| (12, 10) (10, 12) | 46 | 1 |

The jojoba wax ester can be derived from the seed of the jojoba plant (Simmondsia chinensis).

By way of examples of a jojoba wax ester, mention may be made of the product sold under the name Floraesters® 70 by International Flora Technologies Ltd, and the product sold under the name Jojoba Esters-70 by Vantage.

Advantageously, the jojoba ester wax is present in the composition in an amount ranging from 0.1% to 2% by weight and preferably from 0.2% to 0.8% by weight relative to the total weight of the composition.

According to another preferred embodiment, the composition according to the present invention comprises at least one additional hydrocarbon-based wax in addition to the jojoba ester mentioned above.

By way of examples of hydrocarbon-based waxes, mention may be made of beeswax, lanolin wax or Chinese insect wax, rice wax, carnauba wax, candelilla wax, ouricury wax, esparto grass wax, cork fibre wax, sugar cane wax, Japan wax, Berry wax, shellac wax and sumac wax, Helianthus annuus (sunflower) seed wax, montan wax, microcrystalline waxes, paraffins and ozokerite; use may in particular be made of polyethylene waxes, polymethylene waxes, waxes obtained by Fischer-Tropsch synthesis and waxy copolymers, and also esters thereof.

Mention may in particular be made of beeswax, for example the product sold under the name White beeswax BR G889 by Koster Keunen, carnauba wax, for example sold under the name Cerauba T1 Bio by Baerlocher, Helianthus annuus (sunflower) wax sold under the name Sunflower Wax by Koster Keunen, or a mixture thereof.

Preferably, the composition of the present invention comprises Helianthus annuus (sunflower) seed wax.

When it is present, the additional hydrocarbon-based wax is present in the composition in an amount ranging from 0.05% to 5% by weight, preferably from 0.1% to 1% by weight and more preferably from 0.1% to 0.5% by weight, relative to the total weight of the composition.

According to one particular embodiment of the invention, the composition comprises a mixture of glyceryl stearate, sodium steroyl glutamate, cetearyl alcohol, jojoba esters and sunflower seed wax, having the INCI name Cetearyl Alcohol (and) Jojoba Esters (and) Glyceryl Stearate (and) Sodium Stearoyl Glutamate (and) Helianthus Annuus Seed Cera (and) Polyglycerin-3, sold under the name Emulium® Dolcea MB by Gattefosse.

According to one particular embodiment of the invention, the composition comprises:
at least one UV-screening agent;
at least one lipophilic acrylic polymer as defined previously;
at least one C₁₂-C₂₄ saturated fatty alcohol;
at least one C₁₂-C₂₄ fatty acid ester of glycerol;
at least one carboxylic or carboxylate anionic surfactant chosen from amino acids modified with at least one C₈-C₂₄ hydrocarbon-based chain, and salts thereof; and
at least one wax.

According to one preferred embodiment of the invention, the composition comprises:
at least one UV-screening agent;
at least one lipophilic acrylic polymer as defined previously;
at least one C₁₂-C₂₄ saturated fatty alcohol;
at least one C₁₂-C₂₄ fatty acid ester of glycerol;
at least one carboxylic or carboxylate anionic surfactant chosen from amino acids modified with at least one C₈-C₂₄ hydrocarbon-based chain, and salts thereof; and
at least one jojoba ester wax.

Preferably, the composition comprises:
at least one UV-screening agent;
at least one lipophilic acrylic polymer as defined previously;
at least one C₁₂-C₂₄ saturated fatty alcohol;
at least one C₁₂-C₂₄ fatty acid ester of glycerol;
at least one acyl glutamic acid (INCI name: acyl glutamic acid) or a salt thereof (acyl glutamates), the acyl group being a C₁₀-C₃₀, preferably C₁₂-C₂₂, acyl group; and
at least one jojoba ester wax.

According to another particular embodiment of the invention, the composition comprises:
at least one UV-screening agent;
at least one lipophilic acrylic polymer as defined previously;
at least one C₁₂-C₂₄ saturated fatty alcohol;
at least one C₁₂-C₂₄ fatty acid ester of glycerol;
at least one carboxylic or carboxylate anionic surfactant chosen from amino acids modified with at least one C₈-C₂₄ hydrocarbon-based chain, and salts thereof;
at least one jojoba ester wax; and
at least one Helianthus annuus (sunflower) seed wax.

Preferably, the composition comprises:
at least one UV-screening agent;
at least one lipophilic acrylic polymer as defined previously;
at least one C₁₂-C₂₄ saturated fatty alcohol;
at least one C₁₂-C₂₄ fatty acid ester of glycerol;
at least one acyl glutamic acid (INCI name: acyl glutamic acid) or a salt thereof (acyl glutamates), the acyl group being a C₁₀-C₃₀, preferably C₁₂-C₂₂, acyl group;
at least one jojoba ester wax; and
at least one Helianthus annuus (sunflower) seed wax.

According to one particularly preferred embodiment, the present invention proposes a fluid composition for skin care, in the form of an oil-in-water emulsion, comprising, relative to the total weight of the composition:
(i) at least one UV-screening agent;
(ii) from 0.05% to 10% by weight of a lipophilic acrylic polymer comprising monomer units of formulae (A) and (B):

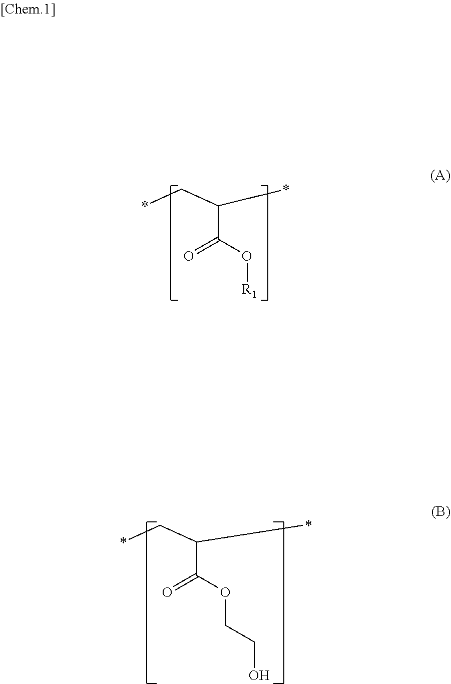

in which:
R₁, independently at each instance, is chosen from alkyl or alkenyl radicals,
and
at least 60% by weight of the R₁ groups are radicals chosen from stearyl and behenyl radicals, the percentage by weight relating to the sum of all the R₁ groups present in the polymer,
and
the weight ratio of the sum of all the hydroxyethyl acrylate units to the sum of all the acrylate units bearing the R₁ group ranges from 1:30 to 1:1,
and the sum of the total of units A and B is at least 95% by weight relative to the total weight of the polymer;
(iii) from 0.2% by weight to 0.8% by weight of at least one jojoba ester wax of formula 3:

R¹—COO—CH₂—R¹ [Chem.3]
where R¹ is CH₃—(CH₂)_{y}—, y is 16, 18, 20 or 22;
(iv) from 0.5% by weight to 2% by weight of at least one C₁₂-C₂₄ saturated fatty alcohol chosen from cetyl alcohol, stearyl alcohol and cetearyl alcohol;
(v) from 0.2% by weight to 0.8% by weight of at least one C₁₂-C₂₄ fatty acid ester of glycerol chosen from glyceryl laurate, glyceryl oleate, glyceryl stearate, glyceryl palmitate, and a mixture thereof; and
(vi) from 0.1% by weight to 0.5% by weight of at least one surfactant chosen from acyl glutamic acids (INCI name: acyl glutamic acid) and salts thereof (acyl glutamates), the acyl group being a C₁₀-C₃₀, preferably C₁₂-C₂₂, acyl group; even more preferentially, it is the stearoyl radical.

### Fatty Phase

The composition in accordance with the invention comprises at least one fatty phase.

The fatty phase can be constituted by all of the fatty substances conventionally used in the cosmetics or dermatological fields; it comprises in particular the wax(es) defined previously and can comprise at least one oil. The fatty phase also comprises the lipophilic screening agent(s), and also the fatty alcohol(s) present in the composition according to the invention.

The term “oil” is intended to mean any fatty substance that is in liquid form at ambient temperature (20-25° C.) and atmospheric pressure (760 mmHg). These oils may be volatile or non-volatile.

For the purposes of the invention, the term “volatile oil” is intended to mean an oil which is capable of evaporating on contact with the skin or the keratin fibre in less than one hour, at ambient temperature and atmospheric pressure. The volatile oil(s) of the invention is/are volatile cosmetic oils, which are liquid at ambient temperature, having a non-zero vapour pressure, at ambient temperature and atmospheric pressure, ranging in particular from 0.13 Pa to 40 000 Pa (10-3 to 300 mmHg), in particular ranging from 1.3 Pa to 13 000 Pa (0.01 to 100 mmHg) and more particularly ranging from 1.3 Pa to 1300 Pa (0.01 to 10 mmHg).

The term “non-volatile oil” is intended to mean an oil that remains on the skin or the keratin fibre at ambient temperature and atmospheric pressure for at least several hours, and that notably has a vapour pressure of less than 10⁻³ mmHg (0.13 Pa).

For the purposes of the present invention, the term “hydrocarbon-based oil” means any oil predominantly comprising carbon and hydrogen atoms, and optionally one or more heteroatoms, in particular nitrogen and oxygen. Thus, these oils can in particular contain one or more ester, ether, fluoro, carboxylic acid and/or alcohol groups.

The term “silicone oil” is intended to mean an oil comprising at least one silicon atom and especially at least one Si—O group.

Mention may notably be made, as non-volatile hydrocarbon-based oils which can be used according to the invention, of:
(i) hydrocarbon-based oils of plant origin, such as glyceride triesters, which are generally triesters of fatty acids and of glycerol, the fatty acids of which can have varied chain lengths from C₄ to C₂₄, it being possible for these chains to be saturated or unsaturated and linear or branched; these oils are in particular wheat germ oil, sunflower oil, grape seed oil, sesame oil, maize oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, red kuri squash oil, pumpkin oil, blackcurrant oil, evening primrose oil, millet oil, barley oil, quinoa oil, rye oil, safflower oil, candlenut oil, passionflower oil or musk rose oil; or alternatively triglycerides of caprylic/capric acids, such as those sold by Stearinerie Dubois or those sold under the names Miglyol 810®, 812® and 818® by Dynamit Nobel;
(ii) synthetic ethers having from 10 to 40 carbon atoms;
(iii) linear or branched hydrocarbons of mineral or synthetic origin, such as liquid petroleum, polydecenes, hydrogenated polyisobutene, such as Parleam, squalane and mixtures thereof;
(iv) synthetic esters, such as the oils of formula RCOOR′ in which R represents the residue of a linear or branched fatty acid comprising from 1 to 40 carbon atoms and R′ represents a hydrocarbon-based chain, in particular branched hydrocarbon-based chain, containing from 1 to 40 carbon atoms, with the proviso that R+R′≥10, such as, for example, Purcellin oil (cetostearyl octanoate), isopropyl myristate, isopropyl palmitate, C₁₂-C₁₅ alkyl benzoate, such as the product sold under the trade name Finsolv TN® or Witconol TN® by Witco or Tegosoft TN® by Evonik Goldschmidt, 2-ethylphenyl benzoate, such as the commercial product sold under the name X-Tend 226® by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name Dub Dis by Stéarinerie Dubois, octanoates, decanoates or ricinoleates of alcohols or of polyalcohols, such as propylene glycol dioctanoate; hydroxylated esters, such as isostearyl lactate or diisostearyl malate; and pentaerythritol esters; citrates or tartrates, such as di(linear C₁₂-C₁₃ alkyl) tartrates, such as those sold under the name Cosmacol ETI® by Enichem Augusta Industriale, and also di(linear C₁₄-C₁₅ alkyl) tartrates, such as those sold under the name Cosmacol ETL® by the same company; acetates;
(v) fatty alcohols which are liquid at ambient temperature, comprising a branched and/or unsaturated carbon-based chain having from 12 to 26 carbon atoms, such as octyldodecanol, isostearyl alcohol, oleyl alcohol, 2-hexyldecanol, 2-butyloctanol or 2-undecylpentadecanol;
(vi) higher C₁₂-C₂₂ fatty acids, such as oleic acid, linoleic acid or linolenic acid;
(vii) carbonates, such as dicaprylyl carbonate, such as the product sold under the name Cetiol CC® by Cognis;
and mixtures thereof.

Among the non-volatile hydrocarbon-based oils which can be used according to the invention, preference will be given more particularly to glyceride triesters and in particular to caprylic/capric acid triglycerides, synthetic esters and in particular diisopropyl adipate, diisopropyl sebacate, isopropyl palmitate, dicaprylyl carbonate, isononyl isononanoate, oleyl erucate, C₁₂-C₁₅ alkyl benzoate, 2-ethylphenyl benzoate and fatty alcohols, in particular octyldodecanol. Preferably, the non-volatile hydrocarbon-based oils are chosen from diisopropyl adipate, diisopropyl sebacate, isopropyl palmitate and dicaprylyl carbonate.

As volatile hydrocarbon-based oils that may be used according to the invention, mention may notably be made of hydrocarbon-based oils containing from 8 to 16 carbon atoms and notably branched C₈-C₁₆ alkanes, such as C₈-C₁₆ isoalkanes of petroleum origin (also known as isoparaffins), such as isododecane (also known as 2,2,4,4,6-pentamethylheptane), isodecane or isohexadecane, the oils sold under the Isopar or Permethyl trade names, branched C₈-C₁₆ esters, isohexyl neopentanoate, and mixtures thereof.

Mention may also be made of the alkanes described in the Cognis patent applications WO 2007/068 371 and WO 2008/155 059 (mixtures of distinct alkanes differing by at least one carbon). These alkanes are obtained from fatty alcohols, which are themselves obtained from coconut kernel or palm oil. Mention may be made of the mixtures of n-undecane (C₁₁) and n-tridecane (C₁₃) obtained in Examples 1 and 2 of application WO 2008/155 059 from Cognis. Mention may also be made of n-dodecane (C₁₂) and n-tetradecane (C₁₄) sold by Sasol respectively under the references Parafol 12-97 and Parafol 14-97®, and also mixtures thereof.

Other volatile hydrocarbon-based oils, such as petroleum distillates, in particular those sold under the name Shell Solt® by Shell, can also be used. According to one embodiment, the volatile solvent is chosen from volatile hydrocarbon-based oils having from 8 to 16 carbon atoms, and mixtures thereof.

The non-volatile silicone oils can be chosen in particular from non-volatile polydimethylsiloxanes (PDMSs), polydimethylsiloxanes comprising alkyl or alkoxy groups, which groups are pendent and/or at the end of the silicone chain and each have from 2 to 24 carbon atoms, or phenylated silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

Volatile silicone oils that may be mentioned, for example, include volatile linear or cyclic silicone oils, in particular those with a viscosity≤8 centistokes (8×10⁻⁶ m²/s) and in particular containing from 2 to 7 silicon atoms, these silicones optionally comprising alkyl or alkoxy groups containing from 1 to 10 carbon atoms. Mention may in particular be made, as volatile silicone oil which can be used in the invention, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and mixtures thereof.

Mention may also be made of the volatile linear alkyltrisiloxane oils such as:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane.

Use may also be made of volatile fluoro oils, such as nonafluoromethoxybutane, nonafluoromethoxybutane, decafluoropentane, tetradecafluorohexane, dodecafluoropentane, and mixtures thereof.

The fatty phase according to the invention can additionally comprise other fatty substances, mixed with or dissolved in the oil.

Another fatty substance which can be present in the oily phase can, for example, be:
a fatty acid chosen from fatty acids comprising from 8 to 30 carbon atoms, other than the fatty-chain amino acids as defined previously, such as stearic acid, lauric acid, palmitic acid and oleic acid;
a gum chosen from silicone gums (dimethiconol);
a pasty compound, such as polymeric or non-polymeric silicone compounds, esters of an oligomeric glycerol, arachidyl propionate, fatty acid triglycerides and their derivatives;
and mixtures thereof.

Preferentially, the overall fatty phase, including all the lipophilic substances of the composition capable of being dissolved in this same phase, including the lipophilic screening agents, represents from 5% to 95% by weight and preferentially from 10% to 80% by weight, with respect to the total weight of the composition.

### Aqueous Phase

The composition in accordance with the invention comprises at least one aqueous phase.

The aqueous phase contains water and optionally other water-soluble or water-miscible organic solvents.

An aqueous phase which is suitable for the invention can comprise, for example, a water chosen from a natural spring water, such as water from La Roche-Posay, water from Vittel, water from Saint-Gervais Mont-Blanc or waters from Vichy, or a floral water.

The water-soluble or water-miscible solvents that are suitable for the invention comprise, in addition to the short-chain alcohols, as defined above, diols or polyols, such as ethylene glycol, 1,2-propylene glycol, 1,3-butylene glycol, hexylene glycol, diethylene glycol, dipropylene glycol, 2-ethoxyethanol, diethylene glycol monomethyl ether, triethylene glycol monomethyl ether, glycerol and sorbitol, and mixtures thereof.

According to one particular embodiment of the invention, the composition comprises an amount of glycerol of less than 10% by weight of the total weight of the composition.

According to a specific form of the invention, the overall aqueous phase, including all the hydrophilic substances of the composition capable of being dissolved in this same phase, represents from 5% to 95% by weight and preferentially from 10% to 80% by weight, with respect to the total weight of the composition.

According to one particular embodiment of the invention, the pH of the composition in accordance with the invention is above or equal to 6.

According to one preferred embodiment of the invention, the pH of the composition in accordance with the invention is between 6.0 and 11.0. Preferably, the pH of the composition in accordance with the invention is between 6.0 and 9.0. Even more preferentially, the pH of the composition in accordance with the invention is between 6.0 and 7.8.

The measurement of the pH of the composition in accordance with the invention can be carried out using a Mettler Toledo MPC227, SevenEasy pH or SevenGo SG2 pH meter, at ambient temperature (25° C.) and atmospheric pressure.

### Cosmetic Active Ingredients

The composition of the present invention may comprise at least one cosmetic active agent.

By way of examples of cosmetic active agents, mention may be made of moisturizing agents such as protein hydrolysates, polyglycerin-3; natural extracts; vitamins such as vitamin A (retinol), vitamin E (tocopherol), vitamin C (ascorbic acid), vitamin B5 (panthenol), vitamin B3 (niacinamide), and derivatives of said vitamins (in particular the esters) and mixtures thereof; urea; caffeine; salicylic acid and derivatives thereof; alpha-hydroxy acids such as lactic acid or glycolic acid, and derivatives thereof; retinoids such as carotenoids and vitamin A derivatives; algal, fungal, plant, yeast and bacterial extracts; enzymes; tightening agents; agents which act on the microcirculation, and mixtures thereof.

According to one particular embodiment of the invention, the composition comprises at least one moisturizing agent. It is preferably polyglycerin-3.

It is easy for those skilled in the art to adjust the amount of cosmetic active ingredient depending on the final use of the composition according to the present invention.

### Additional Adjuvants or Additives

The composition of the present invention can also comprise conventional cosmetic adjuvants or additives, for example fragrances, chelating agents (for example, tetrasodium glutamate diacetate and disodium EDTA), preserving agents (for example chlorphenesin and phenoxyethanol) and bactericides; in addition a surfactant (for example polysorbate 80), co-emulsifier, additional thickeners (such as an acryloyldimethyltaurate homopolymer or an acrylamide/sodium acryloyldimethyltaurate copolymer, an acrylates/C₁₀₋₃₀ alkyl acrylate copolymer, hydroxypropylguar), pH adjusters (for example triethanolamine, citric acid and sodium hydroxide), fillers (for example aluminium starch octenylsuccinate and polymethylsilsesquioxane) and mixtures thereof.

Those skilled in the art can select the amount of additional adjuvants or additives in such a way as to not be detrimental to the final use of the composition according to the present invention.

### EXAMPLES

### Examples of Preparation of Lipophilic Acrylic Polymers According to the Invention

### Determination of the Molecular Weight by Gel Permeation Chromatography (GPC):

The sample is prepared by preparing a solution of the polymer at 10 mg/ml in tetrahydrofuran. The sample is placed in an oven at 54° C. for 10 minutes and then in an oscillating shaker for 60 minutes to aid dissolution. After visual inspection, the sample appeared to be totally dissolved in the solvent.

The sample prepared was analyzed using two polypore 300×7.5 mm columns manufactured by Agilent Technologies, a Waters 2695 chromatographic system, a tetrahydrofuran mobile phase and detection by refractive index. The sample was filtered through a 0.45 μm nylon filter, before being injected into the liquid chromatograph. The standards used for the calibration are the Easi Vial narrow polystyrene (PS) standards from Agilent Technologies.

Polystyrene standards ranging from 2 520 000 to 162 daltons were used for the calibration.

The system is equipped with a PSS SECcurity 1260 RI detector. The polystyrene calibration curve was used to determine the average molecular weight. The recording of the diagrams and the determination of the various molecular weights were performed by the Win GPC Unichrom 81 program.

### Determination of the Melting Point by Differential Scanning Calorimetry (or DSC):

This method describes the general procedure for determining the melting point of polymers by differential scanning calorimetry. This method is based on standards ASTM E791 and ASTM D 34182 and the DSC calibration is performed according to standard ASTM E 9672.

### Example of Preparation of Polymer 1

### Behenyl acrylate/2-hydroxyethyl acrylate copolymer (Polymer 1)

In a 4-necked flask equipped with a side-blade mixer, an internal thermometer, two funnels, a reflux condenser, and an extension for two other necks, 175 g of behenyl acrylate, 25 g of 2-hydroxyethyl acrylate and 0.4 g of 2,2′-azobis(2-methylbutyronitrile) (Akzo Nobel) were added, over the course of 60 minutes at 80° C., to 40 g of isopropanol, with stirring, after having removed the oxygen from the system by means of a nitrogen purge for 20 minutes. The mixture was stirred at 80° C. for 3 hours. The solvent was then removed by vacuum distillation, 1 g of dilauryl peroxide was then added and the reaction was continued for 60 minutes at 110° C. The step was repeated. The mixture was then cooled to 90° C., a stream of demineralized water was added and the mixture was then stirred. The water was removed by vacuum distillation.
Molecular weight: Mn=7300 g/mol, Mw=21 000, Mw/Mn=2.8;
Melting point: 65° C.

### Example of Preparation of Polymer 2

### Stearyl acrylate/2-hydroxyethyl acrylate copolymer (Polymer 2)

In a 4-necked flask equipped with a side-blade mixer, an internal thermometer, two funnels, a reflux condenser, and an extension for two other necks, 155 g of behenyl acrylate, 45 g of 2-hydroxyethyl acrylate and 0.4 g of 2,2′-azobis(2-methylbutyronitrile) (Akzo Nobel) were added, over the course of 90 minutes at 80° C., to 50 g of isopropanol, with stirring, after having removed the oxygen from the system by means of a nitrogen purge for 20 minutes. The mixture was stirred at 80° C. for 3 hours. The solvent was then removed by vacuum distillation, 1 g of dilauryl peroxide was then added and the reaction was continued for 60 minutes at 125° C. The step was repeated. The mixture was then cooled to 90° C., a stream of demineralized water was added and the mixture was then stirred. The water was removed by vacuum distillation.
Molecular weight: Mn=7500 g/mol, Mw=19 000, Mw/Mn=2.6;
Melting point: 49° C.

### Composition Examples

The examples that follow serve to illustrate the invention without, however, being limiting in nature. In these examples, the amounts of the composition ingredients are given as % by weight of starting materials or of active materials, relative to the total weight of the composition.

### Protocol for Evaluating the Screening Efficiency

In vitro: The sun protection factor (SPF) is determined according to the “in vitro” method described by M. Pissavini et al. in International Journal of Cosmetic Science, 40, 263-268 (2018), on the basis of the initial absorbance.

The in vitro UVA protection factor (UVAPF) of a sun protection product against UVA radiation is calculated mathematically by in vitro spectral modelling according to the protocol ISO 24443:2012 (Fr).

Each composition is applied to six rough plates of PMMA, in the form of a homogeneous and even deposit at a rate of 1 mg/cm². Each composition is spread using an automated robot which makes even and uniform movements on three plates termed HD6 (moulded granular plates) and three plates termed SB6 (sandy granular plates). The plate is weighed before and after spreading. Once the spreading has been done on the six plates, the latter are left to stand in Thermo-Masters in the dark at 25° C. for 30 minutes. The measurements are carried out by means of a UV-1000S spectrophotometer from Labsphere. Nine measurements are carried out per plate, then the measurements are analyzed using an Excel spreadsheet which provides the SPF and PPD values of the composition measured.

### Protocol for Evaluating Viscosity

The viscosity is evaluated at 25° C. by means of a Brookfield viscometer. A spindle 2 is used because of the liquid texture of the formula. The viscometer is calibrated, then the spindle is introduced into the product and left to rotate for 10 minutes. The measurement is taken at 30 seconds and at 10 minutes.

### Protocol for Evaluating the Stability of the Compositions of the Invention

Each composition is placed in an incubator at 55° C. for one week in 30 ml bottles. After one week, the compositions are removed and their appearance is analyzed macroscopically.

### Formulation Examples 1 to 4

In these examples, the emulsions are prepared in the following manner.

The aqueous phase A and the fatty phase B are heated separately to 90° C. Once the aqueous phase is completely dissolved, it is stirred at 800 rpm with a rotor stator for 10 minutes for good homogenization. Once the fatty phase is completely clear, the emulsion is then prepared by adding the fatty phase to the aqueous phase. During the addition of the fatty phase, stirring is carried out at approximately 1300 rpm with a rotor stator. Once the entire fatty phase has been added, it is necessary to increase the stirring to 2300-2500 rpm for 10 minutes.

### Example 1-Compositions 1 and 2

The following compositions are prepared (amounts of active material).

TABLE 2

| Phase | Composition | 1 (invention) | 2 (comparative) |
|---|---|---|---|
| A | Aqua | q.s. 100 | q.s. 100 |
| A | Disodium EDTA | 0.10 | 0.10 |
| A | Phenoxyethanol | 0.40 | 0.40 |
| A | Propanediol | 3.00 | 3.00 |
| A | Citric acid | 0.15 | 0.15 |
| A | Glycerol | 7.00 | 7.00 |
| B | Isononyl isononanoate | 3.00 | 3.00 |
| B | Octocrylene | 7.00 | 7.00 |
| B | Ethylhexyl salicylate | 5.00 | 5.00 |
| B | Butyl methoxydibenzoylmethane | 3.00 | 3.00 |
| B | Homosalate | 7.00 | 7.00 |
| A | Caprylyl glycol | 0.30 | 0.30 |
| A | Disodium stearoyl glutamate | 0.25 | 0.25 |
| B | C₁₂₋₂₂ Alkyl acrylate/Hydroxyethyl | 1.50 | 1.50 |
| | acrylate copolymer | | |
| | (Polymer 1) | | |
| B | Tocopherol | 1.00 | 1.00 |
| B | Cetearyl alcohol | 0.61 | 0.61 |
| B | Glyceryl stearate | 0.30 | — |
| B | Jojoba esters | 0.30 | 0.30 |
| B | Sunflower seed wax | 0.12 | 0.12 |
| A | Sodium stearoyl glutamate | 0.12 | 0.12 |

### Results Obtained

The results of the sensory analysis and of the SPF evaluation are as follows:

TABLE 3

| | 1 | 2 |
|---|---|---|
| Composition | (invention) | (comparative) |
| SPF in vitro | 45.6 ± 2.2 | 34.4 ± 0.8 |
| UVAPF in vitro | 23.7 ± 0.9 | 18.8 ± 0.3 |
| Stability (1 week at 55° C.) | stable | unstable |

Composition 1, which comprises the surfactant system in accordance with the invention, exhibits a level of sun protection greater than Composition 2 which does not comprise all the ingredients of the surfactant system according to the invention. Moreover, Composition 1 is stable, which is not the case for composition 2.

### Example 2-Compositions 3 and 4

The following compositions are prepared (amounts of active materials).

TABLE 4

| Phase | Composition | 3 (invention) | 4 (comparative) |
|---|---|---|---|
| A | Aqua | q.s. 100 | q.s. 100 |
| A | Disodium EDTA | 0.10 | 0.10 |
| A | Phenoxyethanol | 0.40 | 0.40 |
| A | Propanediol | 3.00 | 3.00 |
| A | Citric acid | 0.20 | 0.15 |
| A | Glycerol | 7.00 | 7.00 |
| B | Isononyl isononanoate | 3.00 | 3.00 |
| B | Octocrylene | 7.00 | 7.00 |
| B | Ethylhexyl salicylate | 5.00 | 5.00 |
| B | Butyl methoxydibenzoylmethane | 3.00 | 3.00 |
| B | Homosalate | 7.00 | 7.00 |
| A | Caprylyl glycol | 0.30 | 0.30 |
| A | Disodium stearoyl glutamate | 0.30 | 0.25 |
| | (Amisoft HS 21P from Ajinomoto) | | |
| B | C₁₂₋₂₂ Alkyl acrylate/Hydroxyethyl | 1.50 | 1.50 |
| | acrylate copolymer | | |
| | (Tego SP Senstar from Evonik) | | |
| | (Polymer 1) | | |
| B | Tocopherol | 1.00 | 1.00 |
| B | Cetearyl Alcohol (and) Jojoba | 1.5 | — |
| | Esters (and) Glyceryl Stearate (and) | | |
| | Sodium Stearoyl Glutamate (and) | | |
| | Helianthus Annuus Seed Cera (and) | | |
| | Polyglycerin-3 | | |
| | (Emulium Dolcea MB from | | |
| | Gattefosse) | | |
| B | Polyglyceryl-6 distearate (and) | — | 1.5 |
| | Jojoba Esters (and) Cetyl Alcohol | | |
| | (and) Polyglyceryl-3 Beeswax | | |
| | (Emulium Mellifera MB from | | |
| | Gattefosse) | | |

### Results Obtained

The composition stability results are as follows:

TABLE 5

| Composition | 3 (invention) | 4 (comparative) |
|---|---|---|
| SPF in vitro | 45.6 ± 2.2 | 31.0 ± 0.5 |
| UVAPF in vitro | 23.7 ± 0.9 | 17.0 ± 0.3 |
| Stability (1 week at 55° C.) | stable | unstable |

Composition 3, which comprises the surfactant system in accordance with the invention, exhibits a level of sun protection greater than Composition 4 which comprises a surfactant system different from that of the invention. Moreover, Composition 3 is stable, which is not the case for Composition 4.

### Example 3-Compositions 5 to 7

The following compositions are prepared (amounts of active materials).

TABLE 6

| | | 5 | 6 | 7 |
|---|---|---|---|---|
| Phase | Composition | (invention) | (comparative) | (comparative) |
| A | Aqua | q.s. 100 | q.s. 100 | q.s. 100 |
| A | Disodium EDTA | 0.10 | 0.10 | 0.10 |
| A | Phenoxyethanol | 0.40 | 0.40 | 0.40 |
| A | Propanediol | 3.00 | 3.00 | 3.00 |
| A | Citric acid | 0.20 | 0.15 | 0.15 |
| A | Glycerol | 7.00 | 7.00 | 7.00 |
| B | Isononyl isononanoate | 3.00 | 3.00 | 3.00 |
| B | Octocrylene | 7.00 | 7.00 | 7.00 |
| B | Ethylhexyl salicylate | 5.00 | 5.00 | 5.00 |
| B | Butyl methoxydiben- | 3.00 | 3.00 | 3.00 |
| | zoylmethane | | | |
| B | Homosalate | 7.00 | 7.00 | 7.00 |
| A | Caprylyl glycol | 0.30 | 0.30 | 0.30 |
| A | Disodium stearoyl | 0.30 | 0.30 | 0.30 |
| | glutamate | | | |
| | (Amisoft HS 21P from | | | |
| | Ajinomoto) | | | |
| B | Tocopherol | 1.00 | 1.00 | 1.00 |
| B | Cetearyl Alcohol (and) | 1.50 | 3.00 | — |
| | Jojoba Esters (and) | | | |
| | Glyceryl Stearate (and) | | | |
| | Sodium Stearoyl | | | |
| | Glutamate (and) | | | |
| | Helianthus Annuus | | | |
| | Seed Cera (and) | | | |
| | Polyglycerin-3 | | | |
| | (Emulium Dolcea MB | | | |
| | from Gattefosse) | | | |
| B | C₁₂₋₂₂ Alkyl acrylate/ | 1.50 | — | 3.00 |
| | Hydroxyethyl acrylate | | | |
| | copolymer | | | |
| | (Tego SP Senstar from | | | |
| | Evonik) | | | |
| | (Polymer 1) | | | |

### Results Obtained

The sensory analysis results are as follows:

TABLE 7

| | | 6 | 7 |
|---|---|---|---|
| Composition | 5 (invention) | (comparative) | (comparative) |
| SPF in vitro | 45.6 ± 2.2 | 28.1 ± 0.8 | Not measured |
| UVAPF in vitro | 23.7 ± 0.9 | 16.2 ± 0.4 | Not measured |
| Stability (1 week at 55° C.) | stable | unstable | Emulsion im- |
| | | | possible |

Composition 5, which comprises the surfactant system and the lipophilic acrylic polymer in accordance with the invention, exhibits a level of sun protection greater than Composition 6 which does not comprise the lipophilic acrylic polymer according to the invention. Moreover, Composition 5 is stable, which is not the case for Composition 6. As regards Composition 7 which does not comprise the surfactant system in accordance with the invention, since the emulsion was impossible to produce, it cannot be evaluated, in particular in terms of sun protection and of stability.

### Example 4-Compositions 8 and 9

The following compositions are prepared (amounts of active materials).

TABLE 8

| Phase | Composition | 8 (invention) | 9 (comparative) |
|---|---|---|---|
| A | Aqua | q.s. 100 | q.s. 100 |
| A | Disodium EDTA | 0.10 | 0.10 |
| A | Phenoxyethanol | 0.40 | 0.40 |
| A | Propanediol | 3.00 | 3.00 |
| A | Citric acid | 0.20 | 0.20 |
| A | Glycerol | 7.00 | 7.00 |
| B | Isononyl isononanoate | 3.00 | 3.00 |
| B | Octocrylene | 7.00 | 7.00 |
| B | Ethylhexyl salicylate | 5.00 | 5.00 |
| B | Butyl methoxydibenzoylmethane | 3.00 | 3.00 |
| B | Homosalate | 7.00 | 7.00 |
| A | Caprylyl glycol | 0.30 | 0.30 |
| A | Disodium stearoyl glutamate | 0.30 | 0.25 |
| | (Amisoft HS 21P from | | |
| | Ajinomoto) | | |
| B | C₁₂₋₂₂ Alkyl acrylate/Hydroxyethyl | 1.50 | — |
| | acrylate copolymer | | |
| | (Tego SP Senstar from Evonik) | | |
| | (Polymer 1) | | |
| B | Tocopherol | 1.00 | 1.00 |
| B | Cetearyl Alcohol (and) Jojoba | 1.5 | — |
| | Esters (and) Glyceryl Stearate | | |
| | (and) Sodium Stearoyl Glutamate | | |
| | (and) Helianthus Annuus Seed | | |
| | Cera (and) Polyglycerin-3 | | |
| | (Emulium Dolcea MB from | | |
| | Gattefosse) | | |
| B | Glyceryl stearate (and) PEG-100 | — | 3.00 |
| | stearate | | |
| | (Arlacel 165-FP-PA-(RB) from | | |
| | Croda) | | |

### Results Obtained

The sensory analysis results are as follows:

TABLE 9

| Composition | 8 (invention) | 9 (comparative) |
|---|---|---|
| SPF in vitro | 45.6 ± 2.2 | 22.6 ± 2.0 |
| UVAPF in vitro | 23.7 ± 0.9 | 13.3 ± 0.9 |
| Stability (1 week at 55° C.) | stable | slightly unstable |

Composition 8, which comprises the surfactant system and the lipophilic acrylate polymer in accordance with the invention, exhibits a level of sun protection and a stability greater than Composition 9 which is stabilized by another surfactant system.

### Example 5-Compositions 10 to 15-Influence of the pH

The following compositions are prepared (amounts of raw materials).

TABLE 10

| | | 10 | 11 | 12 | 13 | 14 | 15 |
|---|---|---|---|---|---|---|---|
| | | (pH = | (pH = | (pH = | (pH = | (pH = | (pH = |
| Phase | Composition | 4.5) | 5.5) | 6) | 6.5) | 8) | 9) |
| A1 | WATER | 36.25 | 36.25 | 36.25 | 36.25 | 36.25 | 36.25 |
| A1 | TRISODIUM | 0.3 | 0.3 | 0.3 | 0.3 | 0.3 | 0.3 |
| | ETHYLENEDIAMINE | | | | | | |
| | DISUCCINATE | | | | | | |
| A1 | HYDROXYACETOPHENONE | 0.5 | 0.5 | 0.5 | 0.5 | 0.5 | 0.5 |
| A1 | CAPRYLYL GLYCOL | 0.5 | 0.5 | 0.5 | 0.5 | 0.5 | 0.5 |
| A1 | PROPANEDIOL | 3 | 3 | 3 | 3 | 3 | 3 |
| A1 | CETEARYL ALCOHOL (and) | 1.5 | 1.5 | 1.5 | 1.5 | 1.5 | 1.5 |
| | JOJOBA ESTERS (and) | | | | | | |
| | GLYCERYL STEARATE (and) | | | | | | |
| | SODIUM STEAROYL | | | | | | |
| | GLUTAMATE (and) | | | | | | |
| | HELIANTHUS ANNUUS | | | | | | |
| | (SUNFLOWER) SEED WAX (and) | | | | | | |
| | POLYGLYCERIN-3 | | | | | | |
| A1 | SODIUM STEAROYL | 0.5 | 0.5 | 0.5 | 0.5 | 0.5 | 0.5 |
| | GLUTAMATE | | | | | | |
| A1 | GLYCERIN | 7 | 7 | 7 | 7 | 7 | 7 |
| A2 | SODIUM STARCH OCTENYL- | 0.5 | 0.5 | 0.5 | 0.5 | 0.5 | 0.5 |
| | SUCCINATE (and) | | | | | | |
| | HYDROXYPROPYL STARCH | | | | | | |
| | PHOSPHATE | | | | | | |
| A2 | AMMONIUM | 0.45 | 0.45 | 0.45 | 0.45 | 0.45 | 0.45 |
| | POLYACRYLOYLDIMETHYL | | | | | | |
| | TAURATE | | | | | | |
| B1 | OCTOCRYLENE | 7 | 7 | 7 | 7 | 7 | 7 |
| B1 | ETHYLHEXYL SALICYLATE | 5 | 5 | 5 | 5 | 5 | 5 |
| B1 | BUTYL | 3 | 3 | 3 | 3 | 3 | 3 |
| | METHOXYDIBENZOYLMETHANE | | | | | | |
| B1 | HOMOSALATE | 7 | 7 | 7 | 7 | 7 | 7 |
| B1 | C12-22 ALKYL ACRYLATE/ | 1.5 | 1.5 | 1.5 | 1.5 | 1.5 | 1.5 |
| | HYDROXYETHYLACRYLATE | | | | | | |
| | COPOLYMER | | | | | | |
| B1 | C15-19 ALKANE | 5.0 | 5.0 | 5.0 | 5.0 | 5.0 | 5.0 |
| B2 | TOCOPHEROL | 1 | 1 | 1 | 1 | 1 | 1 |
| C | WATER | 20 | 20 | 20 | 20 | 20 | 20 |
| D | CITRIC ACID | 0.25 | 0.072 | 0.046 | / | / | / |
| D | SODIUM HYDROXIDE | / | / | / | / | 0.095 | 0.197 |

### Preparation Method

Weigh out the phases, heat phase A1 in a main tank to 72° C. and additionally heat phase B1 to 72° C. also. Once the temperature is reached, add phase A2 to phase A1 (in the main tank). Add phase B2 to phase B1 just before the emulsification. Introduce phase B (B1+B2) into the main tank, emulsify for 15 minutes. Introduce phase C into the main tank and then cool to 25° C. To reach the target pH, add either citric acid or sodium hydroxide.

### Protocol for Evaluating the pH

Calibrate the Mettler Toledo SevenGo SG2 pH meter with a sufficient amount of buffer solution to ensure the immersion of the electrode (around 2 cm) (buffer pH 7, 4 and then 9, rinsing the electrode with demineralized water between each pH).

Next, measure the pH:
Immerse the tip of the Mettler Toledo InLab Solids Pro SM electrode in the compositions for which the pH is to measured.
Maintain stirring during the measurement so that the glass membrane remains in direct contact with the composition.
Note the pH and temperature values.
Rinse the electrode with demineralized water.
The pH value will be expressed to one decimal place and associated with a temperature (expressed in ° C., with no decimal place).

### Results Obtained

The results obtained after 24 hours at ambient temperature (25° C.) are the following.

TABLE 11

| Composition | 10 (pH = 4.5) | 11 (pH = 5.5) | 12 (pH = 6) |
|---|---|---|---|
| Macroscopic appearance at | Compact cream | Semi-thick cream | Fluid cream |
| t = 0 and at T = 25° C. | | | |
| Microscopic appearance at | Fine emulsion, | Fine emulsion, | Fine emulsion, |
| t = 0 and at T = 25° C. | untidy edges in | clear edges, | clear edges, no |
| | certain zones of the | anisotropic | anisotropy |
| | emulsion, | clusters | |
| | anisotropic flakes | | |
| Viscosity (mPa · s) | 30 s: 4080 | 30 s: 496 | 30 s: 545 |
| at 30 seconds then at 10 | 10 min: 3140 | 10 min: 492 | 10 min: 500 |
| minutes | | | |
| SPF in vitro | 31.6 ± 1.5 | 33.4 ± 1.1 | 43.6 ± 0.5 |
| UVAPF in vitro | 15.7 ± 0.6 | 16.6 ± 0.4 | 20.6 ± 0.3 |
| Stability | Appearance, | Appearance, | Appearance, |
| 1 week at 55° C. | colour, odour: | colour, odour: | colour, odour: |
| | Slight yellowing of | Few points of | Smooth, shiny |
| | the formula, slight | surface syneresis, | and white, |
| | surface syneresis, | not very | odourless |
| | slight “cereal” | pronounced | cream. |
| | odour. | “cereal” odour. | Microscopy: |
| | Microscopy: | Microscopy: | Clear edges, fine |
| | Untidy edges, | Clear edges, fine | and isotropic |
| | anisotropic | and isotropic | emulsion. |
| | clusters. | emulsion. | |

TABLE 12

| Composition | 13 (pH = 6.5) | 14 (pH = 8) | 15 (pH = 9) |
|---|---|---|---|
| Macroscopic appearance | Fluid cream | Fluid cream | Very fluid |
| | | | cream |
| Microscopic appearance | Fine emulsion, | Fine emulsion, | Fine emulsion, |
| | clear edges, no | clear edges, no | clear edges, no |
| | anisotropy | anisotropy | anisotropy |
| Viscosity (mPa · s) | 30 s: 518 | 30 s: 303 | 30 s: 256 |
| at 30 seconds then at | 1 min: 500 | 10 min: 295 | 10 min: 224 |
| 10 minutes | | | |
| SPF in vitro | 43.8 ± 2.2 | 50.7 ± 2.9 | 56.3 ± 1.6 |
| UVAPF in vitro | 20.1 ± 0.8 | 22.4 ± 1.1 | 23.6 ± 0.4 |
| Stability | Appearance, | Appearance, | Appearance, |
| 1 week at 55° C. | colour, odour: | colour, odour: | colour, odour: |
| | Smooth, shiny | Slight surface | Surface |
| | and white, | browning, not | browning, |
| | odourless | very | slight “plastic” |
| | cream. | pronounced | odour. |
| | Microscopy: | “plastic” | Microscopy: |
| | Clear edges, | odour. | Clear edges, |
| | fine and | Microscopy: | fine, very |
| | isotropic | Clear edges, | unsettled and |
| | emulsion. | fine and | isotropic |
| | | isotropic | emulsion. |
| | | emulsion. | |

Compositions 12 to 15, the pH values of which are above or equal to 6, have an improved level of sun protection compared to those of compositions 10 and 11, the pH values of which are below 6.

Compositions 12 to 13, the pH values of which are below 8, have an improved stability compared to compositions 10 and 11, the pH values of which are below 6 and compared to compositions 14 and 15, the pH values of which are above or equal to 8.

1. A composition in the form of an oil-in-water emulsion, comprising:
at least one UV-screening agent;
at least one polymer comprising monomer units of formulae (A) and (B): 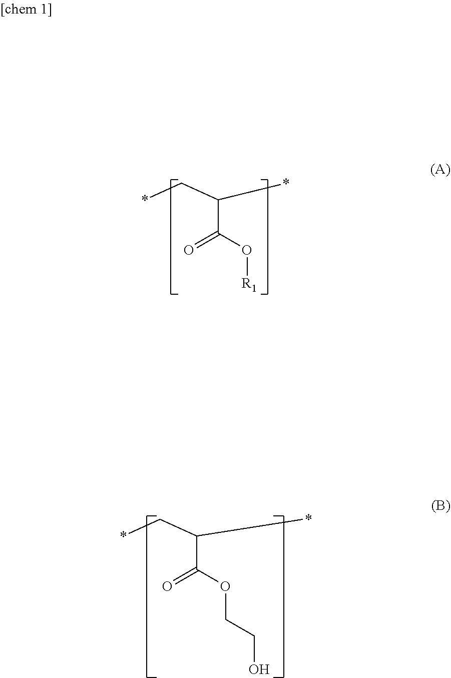
in which:
R₁, independently at each instance, is chosen from alkyl and alkenyl radicals,
and
at least 60% by weight of the R₁ groups are radicals chosen from stearyl and behenyl radicals, the percentage by weight relating to the sum of all the R₁ groups present in the polymer,
and
the weight ratio of the sum of all the hydroxyethyl acrylate units to the sum of all the acrylate units bearing the R₁ group ranges from 1:30 to 1:1;
and the sum of the total of units A and B is at least 95% by weight relative to the total weight of the polymer,
the polymer having a number-average molecular weight Mn ranging from 2000 to 9000 g/mol;
at least one C₁₂-C₂₄ fatty acid ester of a C₂-C₂₄ polyol; and
at least one carboxylic or carboxylate anionic surfactant chosen from amino acids modified with at least one C₈-C₃₀ hydrocarbon-based chain, and salts thereof.

2. The composition according to claim 1, the pH of which is above or equal to 6.

3. The composition according to claim 2, the pH of which is between 6.0 and 11.0.

4. The composition according to claim 1, in which the UV-screening agent(s) is/are chosen from water-soluble organic UV-screening agents, liposoluble organic UV-screening agents, insoluble organic UV-screening agents, inorganic UV-screening agents, and mixtures thereof.

5. The composition according to claim 1, in which the UV-screening agent(s) is/are chosen from liposoluble organic UV-screening agents and mixtures thereof.

6. The composition according to claim 1, in which the amount of the UV-screening agent(s) ranges from 0.1% to 60% by weight relative to the total weight of the composition.

7. The composition according to claim 1, in which, in the lipophilic acrylic polymer, R₁ is constituted of an alkyl radical.

8. The composition according to claim 1, in which, in the lipophilic acrylic polymer, at least 70% by weight of the R₁ groups are behenyl or stearyl radicals.

9. The composition according to claim 1, in which, in the lipophilic acrylic polymer, all the R₁ groups are stearyl or behenyl radicals.

10. The composition according to claim 1, in which, in the lipophilic acrylic polymer, the weight ratio of the sum of all the hydroxyethyl acrylate units to the sum of all the acrylate units bearing the R₁ group ranges from 1:15 to 1:1.

11. The composition according to claim 1 in which the lipophilic acrylic polymer has a number-average molecular weight Mn ranging from 5000 to 9000 g/mol.

12. The composition according to claim 1, in which the lipophilic acrylic polymer has a melting point ranging from 40° C. to 70° C., and preferentially ranging from 45° C. to 67° C.

13. The composition according to claim 1, in which, in the lipophilic acrylic polymer, at least 60% by weight of the R₁ groups are stearyl radicals, and said polymer has a melting point ranging from 40 to 60° C.

14. The composition according to claim 1, in which, in the lipophilic acrylic polymer, at least 60% by weight of the R₁ groups are behenyl radicals, and said polymer has a melting point ranging from 60° C. to 70° C.

15. The composition according to claim 1, in which the lipophilic acrylic polymer(s) is/are present in a content of active material ranging from 0.05% to 10% by weight relative to the total weight of the composition.

16. The composition according to claim 1 in which the C₁₂-C₂₄ fatty acid ester(s) of a C₂-C₂₄ polyol is/are chosen from fatty acid esters of glycerol, and a mixture thereof.

17. The composition according to claim 1, in which the C₁₂-C₂₄ fatty acid ester(s) of a C₂-C₂₄ polyol is/are present in an amount ranging from 0.1% to 2% by weight relative to the total weight of the composition.

18. The composition according to claim 1, in which the carboxylic or carboxylate anionic surfactant(s) is/are chosen from the compounds having the following structure: 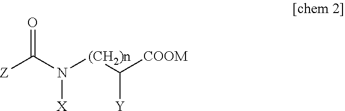
Z represents a linear or branched C₈-C₂₂ alkyl or alkenyl group;
X is a hydrogen atom or a methyl radical;
n is equal to 0 or to 1;
Y is a hydrogen atom, a —(CH₂) 2C(O) OH group or a —(CH₂) 2C(O)O⁻M⁺ group; and
M is a hydrogen atom or a cation.

19. The composition according to claim 1, in which the carboxylic or carboxylate anionic surfactant(s) is/are present in a content ranging from 0.01% to 5% by weight relative to the total weight of the composition.

20. The composition according to claim 1, comprising at least one C₁₂-C₂₄ saturated fatty alcohol, preferably chosen from cetyl alcohol, stearyl alcohol, cetearyl alcohol, myristyl alcohol, lauryl alcohol, tridecyl alcohol, pentadecyl alcohol, hexadecyl alcohol, arachidyl alcohol and behenyl alcohol.

21. The composition according to claim 20, in which the fatty alcohol(s) is/are present in an amount ranging from 0.1% to 5% by weight relative to the total weight of the composition.

22. The composition according to claim 1, comprising at least one wax.

23. The composition according to claim 22, also comprising at least one Helianthus annuus (sunflower) seed wax.

24. A non-therapeutic cosmetic process for caring for and/or making up a keratin material, comprising the application, to the surface of said keratin material, of at least one cosmetic composition as defined in claim 1.
